(19) United States
(12) Patent Application Publication
(10) Pub. No.: US 2009/0221594 A1
(43) Pub. Date: Sep. 3, 2009

(54) STABLE PHARMACEUTICAL COMPOSITIONS OF 5, 10 METHYLENETRAHYDROFOLATE

(76) Inventors: Andrew X. Chen, San Diego, CA (US); Hongjie Wu, San Diego, CA (US); Mark J. Cantwell, San Diego, CA (US); Joan M. Robbins, San Diego, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 12/095,458

(22) PCT Filed: Nov. 30, 2006
(86) PCT No.: PCT/US06/46142

§ 371 Date: Oct. 20, 2008

Related U.S. Application Data
(60) Provisional application No. 60/741,861, filed on Dec. 2, 2005.

(51) Int. Cl. A61K 31/4985

(52) U.S. Cl. 514/249

(57) ABSTRACT

The invention provides a stable lyophilized composition of 5,10 methylenetetrahydrofolate suitable for use in the treatment of cancer and other therapies. The composition comprises 5,10-MTHF in combination with citric acid and ascorbic acid, with the ratio of citric acid to ascorbic acid from about 0.75:1 to about 2.25:1 by weight, and the ratio of total citric acid and ascorbic acid to 5,10-MTHF from about 1.4:1 to about 3.4:1 by weight. Prior to lyophilization, the solution is adjusted to an essentially neutral pH.

### BACKGROUND OF THE INVENTION

Cancer is a major public health concern. Colorectal cancer alone causes approximately 50,000 deaths per year in the United States. Nearly half of the approximately 130,000 cases of colorectal cancer that are diagnosed every year present with or develop into metastatic disease, for which chemotherapy is the only treatment. New effective drug-based therapies for treatment are urgently sought not only for colorectal cancers, but for other cancers such as, for example, breast cancer, pancreatic cancer, gastric cancers, hepatic cancer, bladder cancer, cervical cancer, head and neck cancers, lung cancers, ovarian cancer, and prostate cancer.

The anticancer drug 5-fluorouracil (5-FU) is an inhibitor of thymidylate synthase (TS), an enzyme required for nucleic acid biosynthesis. 5-FU is commonly used to treat cancers such as colorectal and breast cancer, as well as head and neck cancer, pancreatic cancer, stomach cancer, and non-small-cell lung cancer. 5-FU is commonly used in conjunction with folinic acid (FA, leucovorin), which is converted intracellularly into reduced folate, a cofactor for TS. The combination of 5-FU and leucovorin has been found to have increased anti-tumor effects when compared with the use of 5-FU alone.

Unfortunately, the increased anti-tumor effects of 5-FU in combination with leucovorin also involve increased toxicities, such as stomatitis, mucositis, gastrointestinal symptoms, and hematological toxicity, particularly neutropenia, thrombocytopenia, and leucopenia. Such toxicities limit the treatment available to the patient, generally by limiting the dosages of the anti-cancer agent. Thus, there is a need to develop improved anti-cancer drug regimens having reduced toxicity and/or improved antitumor activity that are effective in prolonging survival of the patient.

In addition to the potential for leucovorin to increase the severity of 5-FU systemic toxicity, leucovorin must be intracellularly converted in multiple steps to its active metabolite, 5,10-methylenetetrahydrofolate (variously known by any of the following names: (6R,S)-5,10-methylenetetrahydrofolic acid;
N—[4-(3-amino-1,2,5,6,6a,7-hexahydro-1-oxoimidazo[1,5-f]-pteridin-8(9H)-yl)benzoyl]-L-glutamic acid;
N—[p-(3-amino-5,6,6a,7-tetrahydro-1-hydroxyimidazo[1,5-f]-pteridin-8(9H)-yl)benzoyl]-L-glutamic acid;
(6R,S)-5,10-methylene-5,6,7,8-tetrahydropteroyl-L-glutamic acid;
N5,N10-methylenetetrahydrofolic acid;
D/L-5,10-methylenetetrahydrofolic acid;
(6R,S)-5,10-CH₂—H₄PteGlu-Ca (hereinafter: “5,10-MTHF”)). However, studies have demonstrated that it is possible to use 5,10-MTHF directly (typically, as the calcium salt) and that this active pharmaceutical ingredient has antitumor activity with an apparent safer toxicity profile when used in combination with 5-FU as compared to leucovorin in combination with 5-FU.

The pharmaceutical use of 5,10-MTHF is limited by its instability to various elements, including oxidation by air, neutral and/or acidic environments, chemical degradation, and hydrolysis (M. J. Osborn et al., “The Structure of ‘Active Formaldehyde’,” J. Am. Chem. Soc. 782:4921-4927 (1960)). Because of the desirability to deliver a clinically effective dose of the active form of 5,10-MTHF for the treatment of cancer, it is important that the 5,10-MTHF composition administered to a patient be stable and provide the desired and/or required strength.

It has been taught that 5,10-MTHF can be stabilized with rigorous air occlusion or dissolution in basic pH environments (M. J. Osborn, supra). WO 2004/112761 teaches that a stable pharmaceutical composition of 5,10-MTHF may be obtained by formulating the active ingredient with citrate while adjusting the pH to between 7.5 and 10.5, preferably between 8.5 and 9.5.

### SUMMARY OF THE INVENTION

The inventors of the present invention have now discovered that it is possible to obtain a stable and pharmaceutically active composition of 5,10-MTHF without need to formulate and/or maintain it in a substantially basic pH environment as taught in the prior art. As shown herein, a stable composition of 5,10-MTHF may be formulated wherein the pH of the composition in solution is between about 5 to about 7; prior to lyophilization and for clinical purposes, the stable formulation of 5,10-MTHF in accordance with the present invention may be adjusted to an essentially neutral pH. There is therefore provided in accordance with the present invention a stable lyophilized composition of 5,10-MTHF comprising 5,10-MTHF in combination with citric acid and ascorbic acid, wherein the relative amount of citric acid to ascorbic acid may vary, without substantially affecting the stability of the composition, from a ratio of about 0.75:1 to about 2.25:1 by weight, with the ratio of total citric acid and ascorbic acid to 5,10-MTHF varying from about 1.4:1 to about 3.4:1 by weight. In accordance with a preferred embodiment of the present invention, the ratio of citric acid to ascorbic acid is about 1.5:1 by weight, and the ratio of total citric acid and ascorbic acid to 5,10-MTHF is about 2:1 by weight.

There is also provided in accordance with the present invention a method for formulating a stable, lyophilized and pharmaceutically acceptable composition of 5,10-MTHF, the method comprising the steps of (a) dissolving 5,10-MTHF in a solution containing citric acid and ascorbic acid, wherein the ratio of citric acid to ascorbic acid is from about 0.75:1 to about 2.25:1 by weight, and the ratio of total citric acid and ascorbic acid to 5,10-MTHF is about 1.4:1 to about 3.4:1 by weight; and (b) lyophilizing the solution. The pH of the solution may be between about 5 to about 7. In accordance with a preferred embodiment, prior to lyophilization, the pH of the composition in solution is buffered to an essentially neutral pH.

### DETAILED DESCRIPTION OF THE INVENTION

The present invention provides a novel formulation of 5,10-MTHF which is stable both when in aqueous solution and lyophilized. The formulation of 5,10-MTHF of the present invention may be used as a medicament within a protocol for the treatment of various cancers, in particular, in combination with 5-FU and/or additional chemotherapeutic agents.

The novel formulation of 5,10-MTHF in accordance with the present invention comprises 5,10-MTHF in combination with citric acid and ascorbic acid. The ratio of citric acid to ascorbic acid may vary, without substantially affecting the stability of the composition, from about 0.75:1 to about 2.25:1 by weight, and the ratio of total citric acid and ascorbic acid to 5,10-MTHF may vary from about 1.4:1 to about 3.4:1 by weight. In accordance with a preferred embodiment, the ratio of citric acid to ascorbic acid is about 1-5:1 by weight, and the ratio of total citric acid and ascorbic acid to 5,10-MTHF is about 2:1 by weight. The pH of the solution may vary from about 5 to about 7, with the solution buffered to an essentially neutral pH prior to lyophilization. The formulation in accordance with the invention preferably has osmolality in the isoosmotic range, from about 250 to about 330 mOsm/kg.

The present invention also provides a method of formulating 5,1-MTHF for use as a medicament in the treatment of cancer and other disorders. The method, in accordance with the present invention, comprises the steps of (a) preparing a solution of citric acid and ascorbic acid wherein the ratio of citric acid to ascorbic acid is about 0.75:1 to about 2.25:1 by weight; (b) dissolving 5,10-MTHF in the solution, wherein the ratio of total citric acid and ascorbic acid to 5,10-MTHF is about 1.4:1 to about 3.4:1 by weight; and (c) adjusting and/or buffering the solution to an essentially neutral pH. Preferably, the solution of citric acid and ascorbic acid is chilled to 10° C. and kept chilled at this temperature until all of 5,10-MTHF has gone into solution. In step (c), the essentially neutral pH of the solution is obtained by adjusting and/or buffering the pH of the solution in any manner known in the art, such as with NaOH or HCl. Once all of the 5,10-MTHF has gone into solution, the formulated 5,10-MTHF may then be filled into vials and lyophilized.

### EXAMPLES

### Example 1

### Relative Stability of Various Formulations of 5,10-MTHF

This example shows that 5,10-MTHF formulated with citrate and ascorbic acid, without adjustment to a basic pH (i.e. maintained at an acidic pH of about 5), is more stable that 5,10-MTHF formulated with citrate alone and adjusted to a basic pH of 7.5 or higher.

### Materials

| | | Trisodium | Ascorbic | |
|---|---|---|---|---|
| Test sample | 5,10-MTHF | citrate | acid | pH |
| Nonformulated | 100 mg | 0 | 0 | 6.9 |
| Reference formulation | 100 mg | 269 mg | 0 | 7.5-10.5 |
| lyophile | | | | |
| Test formulation #1 | 100 mg | 250 mg | 176 mg | 5.0 |
| lyophile | | | | |
| Test formulation #2 | 100 mg | 250 mg | 176 mg | 5.1 |
| lyophile | | | | |

Each vial of reference formulation lyophile contains 100 mg 5,10-MTHF, 269 mg sodium citrate dihydrate (trisodium citrate), and pH adjusted to between 7.5 and 10.5 with sodium hydroxide prior to lyophilization.

Each vial of test formulation #1 and #2 lyophile contains 100 mg 5,10-MTHF, 250 mg trisodium citrate, and 176 mg ascorbic acid. The pH was at about 5.

### Dissolution in Water

10 ml sterile water was added to the reference formulation lyophile of 5,10-MTHF and to each of the test formulation lyophiles to give a final concentration of 10 mg/ml (this dissolution volume and concentration corresponds to the most current recommended clinical use guidelines for 5,10-MTHF for cancer therapy). The nonformulated 5,10-MTHF was dissolved in water to a concentration of 6 mg/ml. The lower concentration of nonformulated 5,10-MTHF was due to the fact that nonformulated 5,10-MTHF has a maximum solubility in water of approximately 6 mg/ml (when dissolved in a trisodium citrate, 5,10-MTHF has a much higher (>10 mg/ml) solubility). Samples were stored at room temperature under ambient air conditions to mimic as close as possible expected real-world use.

### Stability Analysis by HPLC

The stability of 5,10-MTHF dissolved in water was measured by HPLC. Data is reported as the normalized purity of 5,10-MTHF based on the starting purity according to the following formula: % Normalized Purity=(% 5,10-MTHF Time X)÷(% 5,10-MTHF Time 0)×(100)

### pH Analysis

The pH of 5,10-MTHF dissolved in water was measured at multiple time points using a digital pH meter.

### Data Analysis

5,10-MTHF stability kinetics were estimated by linear regression analysis (GraphPad Prism software).

### Results

TABLE 1
pH Time Course Following Dissolution in Water

| | | | Test | Test |
|---|---|---|---|---|
| | | Reference | Formulation | Formulation |
| Time | | Formulation | #1 | #2 |
| (Hours) | Nonformulated | Lyophile | Lyophile | Lyophile |
| 0 | 6.88 | 7.89 | 4.96 | 5.08 |
| 0.25 | 6.89 | 7.95 | 5.01 | 5.11 |
| 0.5 | 6.87 | 7.96 | 5.02 | 5.12 |
| 0.75 | 6.85 | 7.95 | 5.02 | 5.12 |
| 1 | 6.83 | 7.93 | 5.02 | 5.11 |
| 2 | 6.77 | 7.87 | 5.01 | 5.11 |
| 4 | 6.80 | 7.80 | 5.01 | 5.1 |
| 6 | 6.83 | 7.74 | 5.03 | 5.13 |
| 8 | 6.81 | 7.70 | 5.03 | 5.13 |
| 24 | 6.79 | 7.59 | 5.04 | 5.13 |

As can be seen from Table 1, pH analysis of the different 5,10-MTHF formulations following dissolution in water revealed variations in starting and steady state pH readings. While nonformulated 5,10-MTHF had an approximately neutral pH of 6.9, the reference formulation lyophile containing only trisodium citrate had a basic pH of approximately 7.9. In contrast, 5,10-MTHF formulated with both ascorbic acid and trisodium citrate had an acidic pH of approximately 5.0 to 5.1. The pH values were stable over a 24 hour period.

TABLE 2
5,10-MTHF Stability Following Dissolution
in Water (% Normalized Purity)

| | | | Test | Test |
|---|---|---|---|---|
| | | Reference | Formulation | Formulation |
| Time | | Formulation | #1 | #2 |
| (Hours) | Nonformulated | Lyophile | Lyophile | Lyophile |
| 0 | 100.0 | 100.0 | 100.0 | 100.0 |
| 0.5 | 94.9 | 98.8 | 99.3 | 97.3 |
| 1 | 92.4 | 97.8 | 99.0 | 97.0 |
| 2 | 90.3 | 96.2 | 98.4 | 96.6 |
| 4 | 85.9 | 90.5 | 96.9 | 95.9 |
| 8 | 77.8 | 86.5 | 95.8 | 95.7 |
| 12 | 69.6 | 80.9 | 95.7 | N/A |
| 18 | 55.1 | 72.4 | 96.6 | 90.8 |
| 24 | 44.9 | 65.0 | 88.9 | 88.8 |

N/A = Data Not Available

As can be seen from Table 2, HPLC analysis showed that each formulation had a different stability profile. Similar to previous reports, nonformulated 5,10-MTHF at neutral pH degraded rapidly over time. 24 hours following dissolution in water, the purity of nonformulated 5,10-MTHF was only 44.9% of the starting purity. The reference formulation lyophile formulated only with trisodium citrate (pH adjusted>7.5) showed slower degradation following dissolution in water. However, purity after 24 hours was still only 65% compared to the starting purity, indicating degradation was not efficiently inhibited by the addition of trisodium citrate and adjustment of pH.

Surprisingly, 5,10-MTHF formulated with both ascorbic acid and trisodium citrate was the most stable formulation, even at an acidic pH which has previously been shown to dramatically decrease 5,10-MTHF stability. This result is even more surprising because it has previously been shown that reducing agents (e.g. 2-mercaptoethanol) cannot efficiently protect 5,10-MTHF from degradation in acidic environments (pH<7).

TABLE 3
5,10-MTHF Linear Regression Degradation Rate Following
Dissolution in Water (% Purity/Hours)

| Formulation | Degradation Rate |
|---|---|
| Nonformulated | −2.2 |
| Reference | −1.4 |
| Formulation Lyophile | |
| Test Formulation #1 | −0.5 |
| Lyophile | |
| Test Formulation #2 | −0.4 |
| Lyophile | |

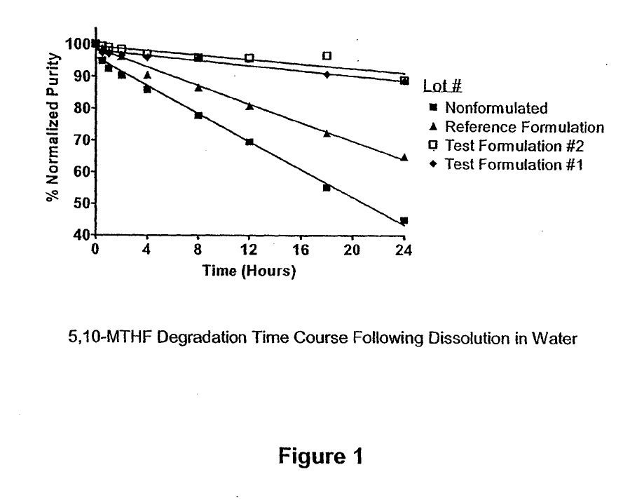

Linear regression analysis of the stability profiles showed that 5,10-MTHF degradation was linear over time (see FIG. 1). The degradation rate (slope of the best-fit line) for each formulation showed the following order, from fastest to slowest degradation rate: nonformulated>formulated with only trisodium citrate>formulated with both ascorbic acid and trisodium citration (Table 3). Moreover, statistical comparison of the best-fit slopes for the reference formulation containing only trisodium citrate to the test formulation containing both ascorbic acid and trisodium citrate showed a statistically slower degradation rate (p<0.05) for the test formulation.

### Example 2

### Formulation of Stable 5,10-MTHF

This example shows a representative method of formulating a stable, lyophilized composition of 5,10-MTHF at an essentially neutral pH comprising citric acid and ascorbic acid.

### Materials

| | mg/100 mL |
|---|---|
| 5,10-MTHF | 5000 |
| Citric acid, Anhydrous, Powder, USP | 6000 |
| Ascorbic acid, Granular, USP | 4000 |
| NaOH/HCl to adjust pH | |
| Water for Injection (WFI), USP to qs | 100 |

### Procedure

1. Sparge WFI with filtered Nitrogen Gas, NF for 30 min.
2. Record tare wt of 100 mL plastic bottle.
3. Weigh out citric acid, ascorbic acid and about 90 g N₂ sparged water. Mix to dissolve.
4. Adjust pH to 7.0±0.1 with 1 N NaOH or HCl.
5. Chill the solution to 10° C.
6. Add 5,10-MTHF, mix to dissolve.
7. Record pH (7.0±0.2).
8. Add more water to 110 g final weight (or 100 mL). Record wt.
9. Pass through a 0.2-micron filter while keeping the solution chilled as possible.
10. Fill into vials (2 mL or 100 mg 5,10-MTHF calcium salt per vial) while keeping the solution chilled as possible.
11. Freeze dry.
12. Seal vials under slight vacuum with nitrogen in the headspace.
13. Crimp the vials.

### Example 3

### Short-Term Stability of Formulated 5,10-MTHF

This example shows that compositions of 5,10-MTHF at an essentially neutral pH, formulated with citric acid and ascorbic acid at varying ratios, are stable in solution for short-term (up to three days).

### Materials

| Formulation | 5,10-MTHF | Citric acid | Ascorbic acid | |
|---|---|---|---|---|
| (Lyophile) | (mg/vial) | (mg/vial) | (mg/vial) | pH |
| F27 | 12.5 | 7.5 | 10 | 7.0 |
| F28 | 12.5 | 15 | 10 | 7.0 |
| F29 | 12.5 | 22.5 | 10 | 7.0 |

### Procedure

1. Add 2.5 mL deionized water to each vial of lyophilized 5,10-MTHF (formulated as described in Example 2) to obtain a 5 mg/mL solution.
2. Chill solution to 2-8 degrees C.
3. At day 0, 1, 2 and 3, withdraw 0.5 g solution and add 4.5 g chilled HPLC diluent (20% dextrose).
4. Inject into HPLC and analyze for concentration.

### Results

TABLE 4
Short Term Stability of Novel Formulations

| | Initial | | Recovery % | | Recovery % | | Recovery % |
|---|---|---|---|---|---|---|---|
| | Conc. | 1 day@5° C. | over initial | 2 day@5° C. | over initial | 3 day@5° C. | over initial |
| Formulation | (mg/g) | (mg/g) | Conc. (mg/g) | (mg/g) | Conc. (mg/g) | (mg/g) | Conc. (mg/g) |
| F27 | 4.15 | 4.14 | 99.7 | 4.16 | 100.2 | 4.1 | 98.7 |
| F28 | 4.60 | 4.62 | 100.3 | 4.63 | 100.6 | 4.6 | 99.9 |
| F29 | 4.61 | 4.37 | 94.6 | 4.34 | 94.0 | 4.4 | 95.4 |

As can be seen from Table 4, all three formulations showed significant stability over the tested time period, with formulation F28 showing the greatest stability.

### Example 4

### Medium-Term Stability of Formulated 5,10-MTHF

This example shows that compositions of 5,10-MTHF, formulated with citric acid and ascorbic acid at varying ratios in accordance with the invention, are stable in lyophilized form for medium-term (7-14 days), even under stress conditions (temperature of 40 degrees C.).

Materials-same as Example 3 above.

### Procedure

1. Maintain lyophile at 40 degrees C.
2. At each time point (1 and 2 weeks), add 2.5 mL deionized water to each vial of lyophilized 5,10-MTHF (formulated as described in Example 2), mixing well for 2 minutes to obtain a clear yellow solution.
3. Withdraw 0.5 g of solution and add 4.5 g chilled HPLC diluent (20% dextrose) to obtain a 0.5 mg/mL theoretical concentration.
4. Inject into HPLC and analyze for concentration.

### Results

TABLE 5
Medium Term Stability of Novel Formulations.

| | Initial | | Recovery % | | Recovery % |
|---|---|---|---|---|---|
| | Conc. | 1 wk@5° C. | over initial | 2 wk@5° C. | over initial |
| Formulation | (mg/g) | Conc. (mg/g) | Conc. (mg/g) | Conc. (mg/g) | Conc. (mg/g) |
| F27 | 4.48 | 4.23 | 94.5 | 4.20 | 93.8 |
| F28 | 4.93 | 4.74 | 96.2 | 4.88 | 99.1 |
| F29 | 4.97 | 4.80 | 96.5 | 4.78 | 96.1 |

As can be seen from Table 5, all three lyophilized formulations were stable after two weeks, with formulation F28 showing the greatest stability.

### Example 5

### Long-Term Stability of Formulated 5,10-MTHF

This example shows that the composition of 5,10-MTHF at an essentially neutral pH, formulated with citric acid and ascorbic acid, in accordance with the present invention, is stable in lyophilized form for long-term, even when maintained at a temperature of 25 degrees C.

### Materials.

Formulated and lyophilized 5,10 MTHF was prepared as described above. Each vial contained 100 mg 5,10-MTHF, 127 mg citric acid and 85 mg ascorbic acid.

### Procedure.

Lyophiles were maintained either at 5 degrees C. or at 25 degrees C. and 60% relative humidity. At each time point (three weeks and six weeks), 10 mL of sterilized water was added to each vial of lyophilized 5,10-MTHF, and a clear, light amber solution was obtained, and pH measured. 2 mL of solution were further diluted with 25 mL HPLC diluent, and analyzed for concentration by HPLC.

### Results.

TABLE 6
Long Term Stability of Formulated 5,10-MTHF

| | | | | | | Total |
|---|---|---|---|---|---|---|
| Storage | | | Solution | | Recovery | Impurities |
| Condition | Time | Cake Appearance | Appearance | pH | mg | (UV area %) |
| | t 0 | uniform cake, light beige/orange | clear, light | 7.13 | 107.5 | 5.3 |
| | | with few specks of dark orange | amber | | | |
| | | on top | | | | |
| 5° C. | 3 wk | uniform cake, yellow-orange | clear, light | 7.17 | 93.9 | 5.5 |
| | | cake w/small specks of darker | amber | | | |
| | | orange on top | | | | |
| 25° C. | 3 wk | uniform cake, light beige/orange | clear, light | 7.13 | 92.4 | 5.9 |
| | | with few specks of dark orange | amber | | | |
| | | on top | | | | |
| 5° C. | 6 wk | uniform cake, light beige/orange | clear, light | 7.17 | 93.4 | 5.5 |
| | | with few specks of dark orange | amber | | | |
| | | on top | | | | |
| 25° C. | 6 wk | uniform cake, light beige/orange | clear, light | 7.15 | 94.1 | 5.4 |
| | | with few specks of dark orange | amber | | | |
| | | on top | | | | |

As can be seen from Table 6, the formulation was stable after six weeks, at both storage conditions.

1. A stable, lyophilized composition of 5,10-MTHF comprising citric acid and ascorbic acid or pharmaceutical salts thereof, wherein the ratio of citric acid to ascorbic acid is in the range of about 0.75:1 to about 2.25:1 by weight and the ratio of total citric acid and ascorbic acid to 5,10-MTHF is in the range of about 1.4:1 to about 3.4:1 by weight.

2. The composition of claim 1 wherein the ratio of citric acid to ascorbic acid is about 1.5:1 by weight.

3. The composition of claim 1 wherein the ratio of total citric acid and ascorbic acid to 5,10-MTHF is about 2:1 by weight.

4. The composition of claims 1-3 in solution wherein the solution has a pH of about 5 to about 7.

5. The composition of claim 4 wherein the solution has an essentially neutral pH.

6. A method of formulating a stable, lyophilized composition of 5,10-MTHF, the method comprising the steps of (a) preparing a solution of citric acid and ascorbic acid or pharmaceutical salts thereof, wherein the ratio of citric acid to ascorbic acid is in the range of about 0.75:1 to about 2.25:1 by weight; (b) dissolving 5,10-MTHF in the solution, wherein the ratio of total citric acid and ascorbic acid to 5,10-MTHF is in the range of about 1.4:1 to about 3.4:1 by weight; and (c) lyophilizing the solution.

7. The method of claim 6 wherein the ratio of citric acid to ascorbic acid is about 1.5:1 by weight.

8. The method of claim 6 wherein the ratio of total citric acid and ascorbic acid to 5,10-MTHF is about 2:1 by weight.

9. The method of claims 6-8, further comprising the step of adjusting the solution to an essentially neutral pH prior to lyophilization.
